(19) United States
(12) Patent Application Publication
(10) Pub. No.: US 2014/0332011 A1
(43) Pub. Date: Nov. 13, 2014

(54) ORAL SLEEP APNOEA DEVICE

(71) Applicant: HIBOBI PTY LTD, New South Wales (AU)

(72) Inventor: Gregory Turek, New South Wales (AU)

(21) Appl. No.: 14/365,030

(22) PCT Filed: Dec. 17, 2012
(86) PCT No.: PCT/AU2012/001546

§ 371 Date: Jun. 12, 2014

(30) Foreign Application Priority Data
Dec. 16, 2011 (AU) 2011254095

(51) Int. Cl. A61F 5/56

(52) U.S. Cl. CPC A61F 5/566
USPC 128/848

(57) ABSTRACT

An oral treatment device for sleep apnoea and snoring, including a pad adapted to rest under the tongue of a user, so as to support the tongue from below, and engage the lower surface of the tongue using a reflex action of the relaxed tongue, the tongue flow adaptation effect. Implementations also include textured surfaces on the front of the device to encourage the tongue-tongue reflex and tongue protrusion.

The device is designed to be tolerated for relatively long periods of time, and minimise any disruption to sleep, while providing effective tongue positioning.

### TECHNICAL FIELD

The present invention relates to oral devices for the treatment of obstructive sleep apnoea (OSA), snoring and related disorders.

### BACKGROUND TO THE INVENTION

OSA is a chronic condition which affects many sufferers around the world. One treatment which has been applied is the continuous positive air pressure (CPAP) system, in which a mask and associated pump is used to apply positive air pressure to the airway in order to prevent its collapse. Whilst CPAP is effective in many cases, it suffers from an issue of poor on-going compliance from patients.

Another approach has been to use oral appliances of various kinds. Mandibular advancement splint (MAS) devices hold the lower jaw forward, so as to better open the airway, and keep the base of the tongue forward from the airway opening. Holding the lower jawbone forward helps open up the pharyngeal space above the airway, however, in most cases it is the base of the tongue falling back that blocks the pharyngeal airway. Holding the lower jaw forward exerts some forward influence on the resting position of the tongue. However the tongue is only loosely connected to the lower jaw bone, so holding the lower jaw forward does not necessarily hold the tongue far enough forward to prevent airway obstruction.

MAS devices are very well tolerated, however, they have proved most effective in cases of snoring and mild OSA, particularly when patients have relatively low BMI. In more severe OSA and higher BMI patients, MAS devices have proved less effective.

Other devices have been proposed which operate by directly gripping the tongue. For example, US Patent application No. 2009/0126742 by Summer discloses a tongue gripping and restraining device having a surface with relatively sharp protrusions which mechanically squeezes the tongue from both sides. Other tongue control devices are disclosed, for example, in U.S. Pat. No. 5,988,170 to Thomas, and CA2206701 to De Voss. The disclosures of these references are hereby incorporated by reference.

It is an object of the present invention to provide oral appliances including tongue positioning structures which provide a wider range of effectiveness for snoring, OSA and related disorders than existing devices.

### SUMMARY OF THE INVENTION

Broadly, the present invention uses a recognition that the tongue is responsive to suitably positioned passive structures to assist in correct positioning of the tongue when the user is asleep or unconscious. A passive oral device can assist the tongue in maintaining a desired position in the user's mouth when the user is asleep, so as to improve the function of the airway. In particular, these devices primarily engage the tongue from below, and present a specifically shaped and textured surface to the lower surface of the tongue.

In one form, the present invention provides a device including a lower support which engages the lower teeth, and which includes a tongue engaging region, which operatively sits under the tongue, the region being shaped and positioned so that the tongue flows over and around the region, so that the tongue tends to remain in contact with the region. The tongue engaging region may be integral with the lower support, or may be carried by a resilient member.

The device may in some implementations further include an upper support which engages the upper teeth, the upper and lower supports being configured as a mandibular advancement device. The upper support may additionally include one or more surfaces to assist in retaining the tongue in contact therewith by engaging the upper surface of the tongue.

According to one aspect, the present invention provides an oral device, including a lower carrier adapted to be positioned over the teeth of a user, the carrier including an integrally formed tongue support for supporting the tongue from below. In one form, the device includes suitably textured surfaces to assist the tongue in resting in the desired position in the mouth, so as to maintain an effective airway.

According to another aspect, the present invention provides a method for assisting the tongue to adopt a desired position in the mouth of a user, including the steps of providing an oral device including a region shaped and textured so that the tongue tends to remain in contact therewith, the region being positioned so that the tongue tends to remain in the desired position when the user is asleep.

According to another aspect, the present invention provides a tongue positioning device including a lower support, an upper support, an upper tongue engaging member, and a lower tongue engaging member, the lower support and upper support being operatively biased towards each other the upper and lower tongue engaging members being held in a floating suspension, so that a similar gripping force is applied to the tongue of a user by the upper and lower tongue engaging members when the tongue is positioned through a range of vertical positions.

According to another aspect, the present invention provides a method for providing tongue support in an oral device, including resiliently supporting the tongue between upper and lower supports, the upper and lower supports being operatively biased toward each other, so as to provide a floating suspension for the tongue.

According to another aspect, the present invention provides a method of providing a device which is adapted to provide tongue support, including the steps of providing a mandibular advancement device, said mandibular advancement device including a lower support which is shaped and positioned so that the tongue flows over and around the upper surface of the support, so that the tongue tends to remain in contact with the region.

According to another aspect, the present invention provides a tongue positioning device including a lower support, an upper support, and a lower tongue engaging member, the lower support and upper support being operatively biased towards each other, the lower tongue engaging member being resiliently biased towards the upper support, so that that operatively the tongue is urged towards the upper support and generally the tongue is urged to a position so that operatively, when the user is asleep, the airway of a user remains operative.

According to a further aspect, the present invention provides a method for providing tongue support in an oral device, including resiliently supporting the tongue from below using a resiliently mounted lower supports pad, the lower support pad being operatively biased toward the roof of a user's mouth.

Implementations of the present invention provide advantages in effective positioning of the tongue, so as to maintain an effective airway, while minimising the discomfort and risk of injury for the user.

### BRIEF DESCRIPTION OF THE DRAWINGS

Illustrative embodiments of the present invention will now be described with reference to the accompanying figures, in which:

FIG. 1 is a view, partly in section, of one implementation of the present invention;

FIG. 2 is a view similar to FIG. 1, including the tongue;

FIG. 3 is a perspective view showing the upper carrier and pad from below according to one implementation;

FIG. 4 is a view showing the upper carrier and pad from above according to one implementation;

FIG. 5 is a perspective view showing the lower carrier and pad from below according to one implementation;

FIG. 6 is a plan view of the fitted carriers, pads and tongue from above;

FIG. 7 is a side view of a lower pad;

FIG. 8 is a front perspective view showing the assembled device;

FIG. 9 is a rear perspective view of assembled device of FIG. 8;

FIG. 10 is a view of an alternative lower pad;

FIG. 11 is a view of another alternative lower carrier;

FIG. 12 is a view of the central part of another alternative upper carrier; and

FIG. 13 illustrates the carriers of FIGS. 11 and 12 together.

### DETAILED DESCRIPTION

The present invention will be described with reference to particular illustrative examples. However, it will be understood that the examples are illustrative of possible implementations of the invention, and not intended to be limitative of the scope of the present invention. Further, several alternative implementations are disclosed, which in some cases may be used in combination, or used sequentially in order to achieve a desired patient outcome. It will be appreciated that further alternative implementations using the principles of the present invention are possible and contemplated by the inventor.

It has been recognized by the present inventor that it is critical not only to ensure that the tongue does not move to an obstructive position during sleep but also to ensure that the tongue positioning device is both safe and comfortable for the user. No device, however effective, will achieve long term user compliance unless it is well tolerated and provides only a minimum of discomfort for the user. The device must be tolerated for relatively long periods of time, and minimise any disruption to sleep. Implementations of the present invention accordingly provide simple devices, which provide effective tongue positioning. These implementations are able to provide an effective treatment device and method for OSA and related conditions.

In order to better understand the operation of implementations of the present invention, it is important to consider the behaviour of the tongue. The tongue is a difficult organ to restrain because it is very sensitive, slippery and, most importantly, variable in size, shape and mechanical properties. The underside of the tongue is a very delicate mucous membrane, while the upper surface is a much tougher yet still sensitive surface. Gripping either surface with any kind of projection or angled edge, especially against the underside of the tongue, will inevitably create discomfort and potential injury.

The tongue is a muscular organ, with a complex set of reflexes and autonomous behaviours. It performs many complex tasks without conscious control. Failure to recognise and take account of the complex characteristics of the tongue when designing and implementing oral devices is likely to lead to unsatisfactory outcomes.

One aspect of tongue behaviour which has been newly identified by the inventor is tongue flow adaptability, or TFA. When the tongue is placed over another surface in a relaxed state, the surface of the tongue flows in a fluid-like way so as to intimately conform its surface to the external surface. It will flow over, around and grip the surface. It is this TFA feature which provides the surfaces according to implementations of the present invention with the ability to become enveloped by tongue tissues and thus to hold the corresponding tongue surface. Conventional gripping or grasping is unnecessary. In some implementations and for some users, it may be necessary to exert enough force on the tongue to ensure that the TFA effect is operative so as to resist passive or unconscious tongue withdrawal, for example by using a floating suspension.

The degree of restraint provided according to implementations of the present invention, which will be further explained below, are not intended to resist a conscious withdrawal of the tongue, only to resist the force of gravity and to maintain adequate engagement between the device and the tongue in the event of changes in the size and consistency of the tongue. During sleep, when the tongue generally becomes softer and smaller.

In one set of embodiments of the present invention, pads or surfaces are provided which are intended to be engaged by the lower surface of the tongue, and so exhibit location of the tongue with the mouth using only the engagement of the tongue with the pad or surface using the TFA effect. These embodiments also generally include an upper component and a mandibular advancement device.

For some patients, this effect alone may not provide sufficient support. Another implementation of the present invention maintains a relatively constant engagement force with both the upper and the lower surface of the tongue. This engagement allows for the engagement of the pads with the upper and lower tongue surfaces to provide an adequate resistance to removal in many cases.

Some users will be adequately managed using a lower pad or surface alone, which may be directly or resiliently attached to a lower support, optionally with an upper support. Others will require additional assistance in restraining the tongue, at least initially, by utilising upper and lower support pads, one or more of which may be resiliently supported.

Any restraining system which operates from both the upper and lower tongue surfaces, and which does not have such a dynamic engagement structure, illustratively provided by the floating suspension of the implementations discussed below, must grip or grasp with an initially excessive force to remain in contact when the tongue has reduced in thickness and tone during sleep. This relatively tight grip leads to inevitable comfort and safety issues.

A critical factor in tongue restraint is the necessity to allow the tongue to be instantly withdrawn from the device without trauma. The tongue is very powerful and flexible, so attempts to hold it with sufficient force to resist all attempts of withdrawal are virtually impossible, because even if the action results in trauma, one can pull ones tongue out of virtually any entrapment. Sudden events such as sneezing, coughing or yawning require instant reflex tongue withdrawal. Any method of gripping or holding the tongue which does not allow this instant involuntary reflex withdrawal will inevitably result in trauma upon sudden withdrawal. Devices using surfaces with angular or protruding gripping elements are likely to traumatize the tongue if it is suddenly withdrawn.

Another aspect of tongue behaviour is the tongue-tongue reflex. When a pressure or touch is applied to the tip and surrounding areas of the tongue, the tongue orients itself towards that stimulus. The stronger the stimulus, the greater the probability that the tongue will move towards the stimulus. Further details regarding tongue reflexes can be obtained from the literature, for example from Moses, ‘Dentist's treatment of snoring’, Sleep Diagnosis and Therapy, vol 2 no 6 December 2007, the disclosure of which is hereby incorporated by reference.

Thus, it will be understood that the present invention contemplates the use of a family of potential devices, in most implementations with customisation and selection under the supervision of a suitable clinician to ensure that adequate airways are maintained during sleep, while preferably limiting any intervention to the minimum required to achieve the desired result.

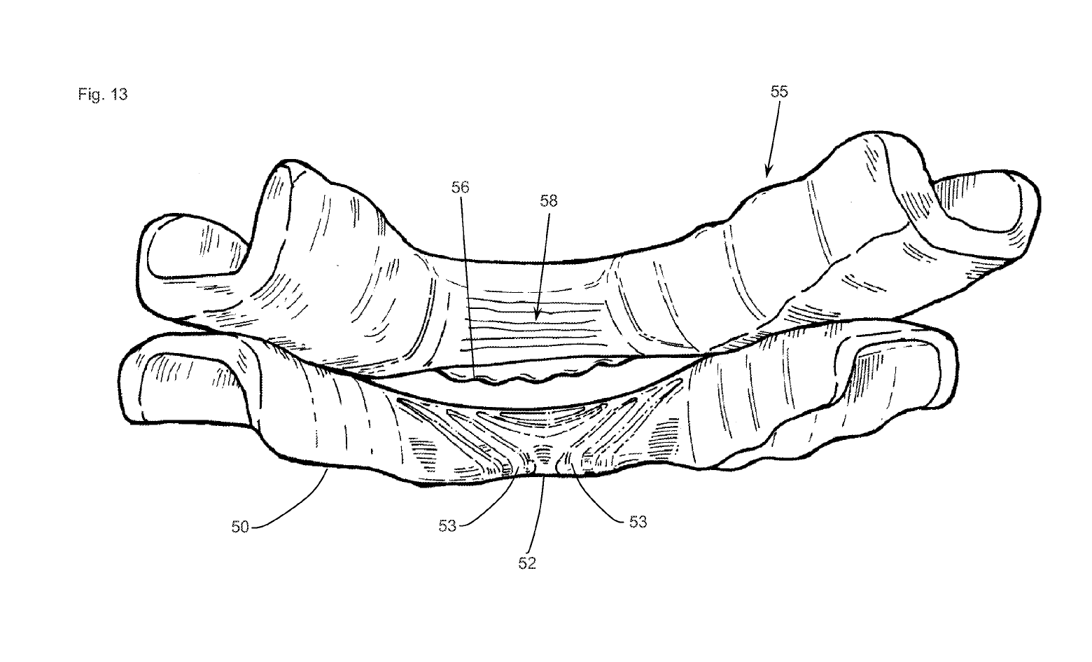

One implementation of the present invention, using a lower pad within the context of a mandibular advancement device, will now be described with reference to FIGS. 11, 12 and 13.

Lower splint 50 according to this implementation is in its basic form, material and fitting comparable to the lower carrier 11 described below. However, it also includes an integral pad 51 extending inside the curve of the teeth. The pad includes a groove 52 which operatively is intended to receive the lingua frenum (not shown). Thus, the pad in use sits well back onto the tongue, not just near the tip but well under the rear portion of the tongue. As such, the TFA effect is given a relatively large surface area on which to allow the tongue to interact with the surface. Further, the tongue is supported further back and urged into a more forward position, so that the airway is better held open. This placement well back onto the tongue, although with an engagement from the front of the tongue, is facilitated by the groove 52, which operatively receives the lingue frenum to allow the tongue to protrude relatively more than would be possible with a pad without a groove 52.

The pad 51 surfaces include a series of generally parallel grooves 53. These caught alternatively be differently shaped features if desired, for example small ‘pimple’ like protrusions, differently shaped grooves, or any other suitable surface texture features. These features according to the present implementation are ideally channels or trenches of the order of 2 mm wide and 2 mm deep, with slightly rounded edges. Any suitable texturing which is effective could be used.

It is not easy to test, because as the tongue rests and relaxes, it flows in a fluid like way into the grooves or channels providing retention. As soon as one tries to test this retentive effect, even by just slightly pulling the tongue back, the tongue slightly tenses up and is out of the grooves. One way to test the textured surface is for the user to lie on back with their tongue as relaxed as possible over the texture (grooves), then feel how much resistance there is to tongue falling back, however, importantly without any conscious pull on tongue. Any texture which facilities the operation of the device, by operative of the TFA effect, could be used, and it is anticipated that alternative textures could be used.

The texture in the pad 51 should allow as much surface area contact with the tongue underside as possible. The channels should be large and robust enough in width and depth to maximise retentive effect. If the texture is composed of features which are too fine, the retentive ability will be degraded by the thick mucous film secreted under tongue. In other words, fine ripples will be obliterated by the mucousy saliva film.

The pad is also angled so as to provide an inclined front ramp 54, (which in alternative implementations can be with or without a textured surface). This ramp may be omitted in some implementations. The ramp is formed by the inclination of the pad 51. It could be more horizontally placed but by being angled slightly downward (facing back) it follows the natural forward trajectory and shape of the tongue underside as it slides forward and engages over the pad surface.

Upper splint 55 is, again, generally similar in materials and form to upper carrier 10, as described in more detail below. It further includes a textured surface 58 and edge 56 which are intended to be engaged by the tongue and assist in the tongue correctly locating on the splint 55. A further feature of the upper carrier is that the inside front portion 57 is generally shaped as an elliptical cone, so as to receive the tongue when protruded forward. Upper splint 55 mainly provides the stimulating textured surfaces 56 and 58 which the tongue tip operatively “plays with”, thus learning where the reference points are for correct forward posture. The textured surfaces 56, 58 also functions to stimulate the Tongue-Tongue protrusive reflex. The top splint, as noted, also provides the top half of the tongue elliptical cone shaped enclosure 57 (tongue tip space).

One advantage this implementation has relative to the implementations to be discussed below, with a resiliently supported pad, is that this embodiment generally requires a smaller vertical opening between the upper and lower splints, as there is no need to leave room to accommodate the volume occupied by the pads. This has the advantage that a larger vertical opening causes an increased tendency (in at least some users) for the for the tongue to rotate backwards—i.e. towards the rear of the mouth, and this rotation causes degradation of the airway.

Any system of tongue restraint involving gripping elements will prevent natural tongue posturing because once the tongue is pulled out of restraint during sleep, for example by coughing, it cannot unconsciously find its way back through a complex series of manoeuvres into the restraint. The tongue's reflexive response is to protrude forward against whatever surface is there. The integral pad shape on the lower splint being inclined slightly downwards, provides a natural ramp like surface for the tongue to easily slide up and forward.

Hence, this implementation of the present invention is believed to be effective by mainly training or conditioning the tongue, using reflex stimulation, and using TFA for added restraining effect.

It will be appreciated that this implementation lends itself well to CAD manufacture, for example using conventional 3D printing such as is used by Resmed Limited with their Narval device. Alternatively, conventional dental appliance manufacturing techniques and material may be used. It will be appreciated that implementations of the present invention, in which a tongue support structure is integrated into a lower splint, may be developed with most existing oral sleep devices.

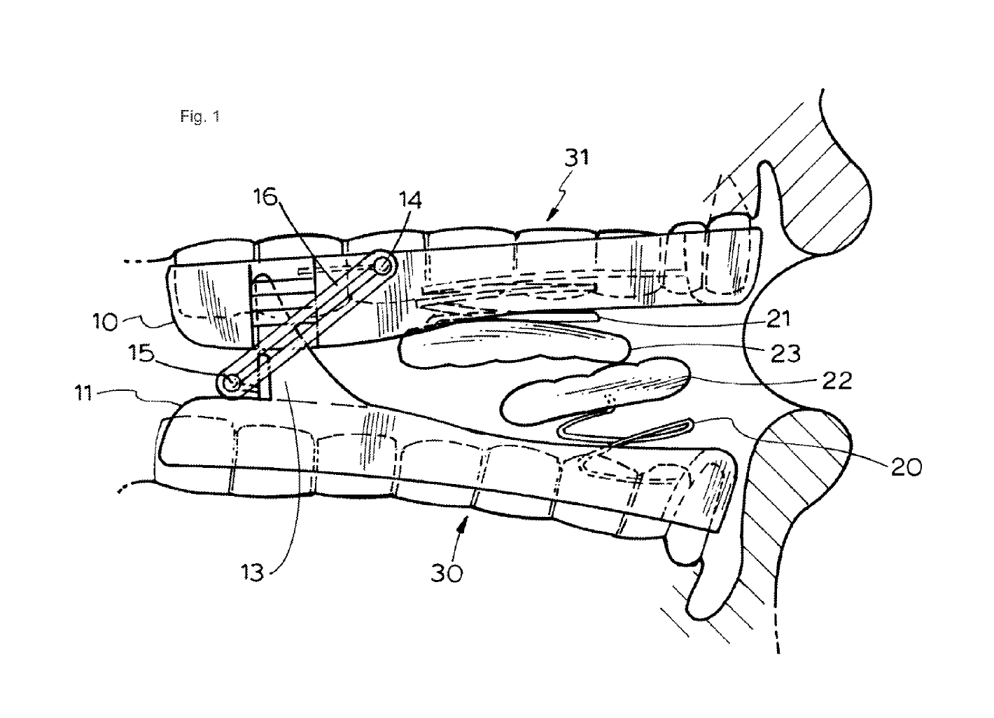

Another aspect of the present invention will now be described in more detail, with reference to FIG. 1. A device according to this implementation includes an upper carrier base 10, and a lower carrier base 11. In a preferred implementation, upper carrier base 10 and lower carrier base 11 may be provided by a conventional MAS device, specified, fitted and made in a conventional way, other than as we will describe below. For example, the MAS device may be a device such as that commercially available as the Somnodent from Somnomed Limited, the Moses appliance, distributed in Australia by Southern Cross Dental Laboratory, the Silent Night device available commercially from many dental laboratories around the world, or a monobloc type device. Alternatively, it could be mounted on a passive, non-MAS type splint or similar device, although this is not preferred.

The upper carrier base 10 is located and moulded to fit the upper teeth 31, and the lower carrier base 11 is located and moulded so as to fit the lower teeth 30. It would be possible to provide a less customised carrier, similar to a mouthguard, and achieve some of the advantages of the present invention. However, it is preferred that a customised, moulded splint, preferably based upon a correctly taken impression from a dentist, is used. As with conventional MAS splints, the upper and lower carriers are biased towards each other using appropriate elastics 16, attached to lugs 14, 15. The elastics are tensioned only sufficiently to resist passive opening of the jaw by gravity, active voluntary jaw opening is not prevented by the elastics.

Lower carrier 11 is also attached to a helical spring 20, which is in turn attached to a lower pad 22. Upper carrier is similarly attached to a helical spring 21, which is in turn connected to an upper pad 23. The upper and lower pads 21, 22 can be formed from any suitable biocompatible material, for example a hard dental plastic such as PMMA or polycarbonate. They should be formed so as to have smooth curve and edges, and preferably to have a pattern of rounded grooves or similar shapes on the surface. This assists in appropriate engagement with the tongue, so as to improve the retaining force provided by the TFA effect described above.

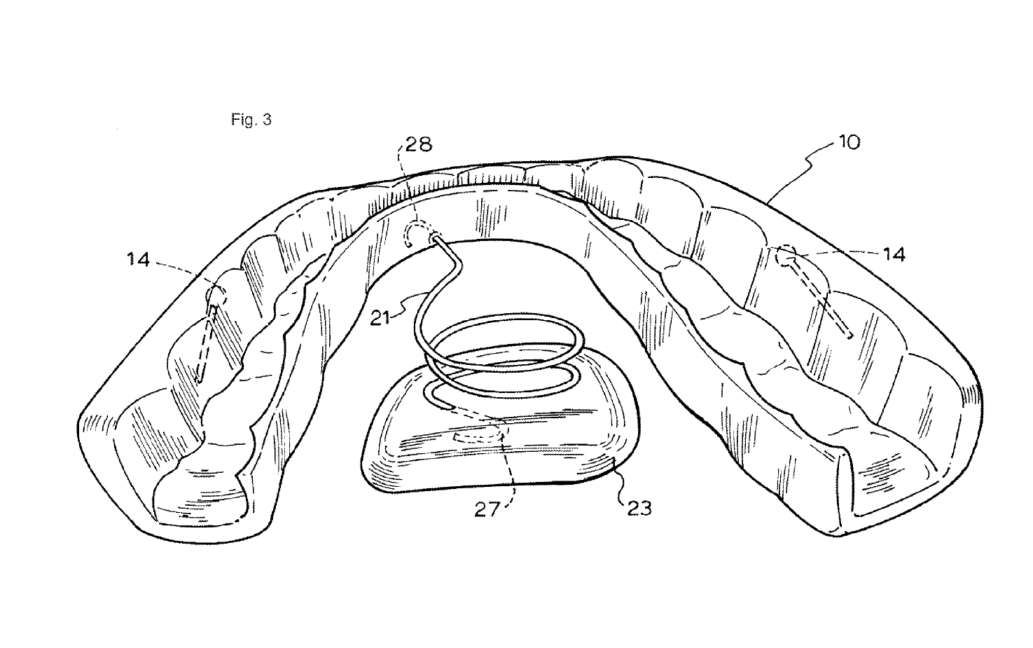

In the form illustrated, which can be better seen from FIGS. 3, 4 and 5, the pads are both of generally trapezoid shape, with the shortest side oriented to the front of the mouth in use. The upper pad 23 is approximately 28 mm long and 28 mm wide at its widest extent. The lower pad 22 is approximately 25 mm long and 27 mm wide at its widest point. Upper pad 23 is operatively required to be able to move within the palatal arch 29. It must be sized, shaped and affixed to the spring 21 so that it can move freely over a suitable range, as the spring is compressed, within the internal surfaces of the carrier. This ability to move freely within the required range is important for the effective operation of this implementation of the device.

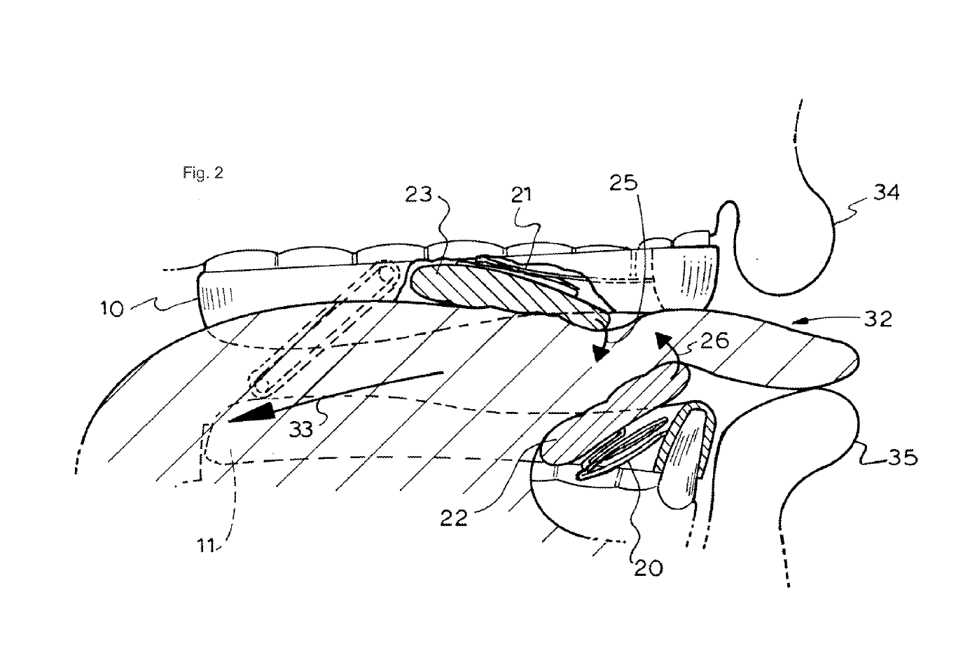

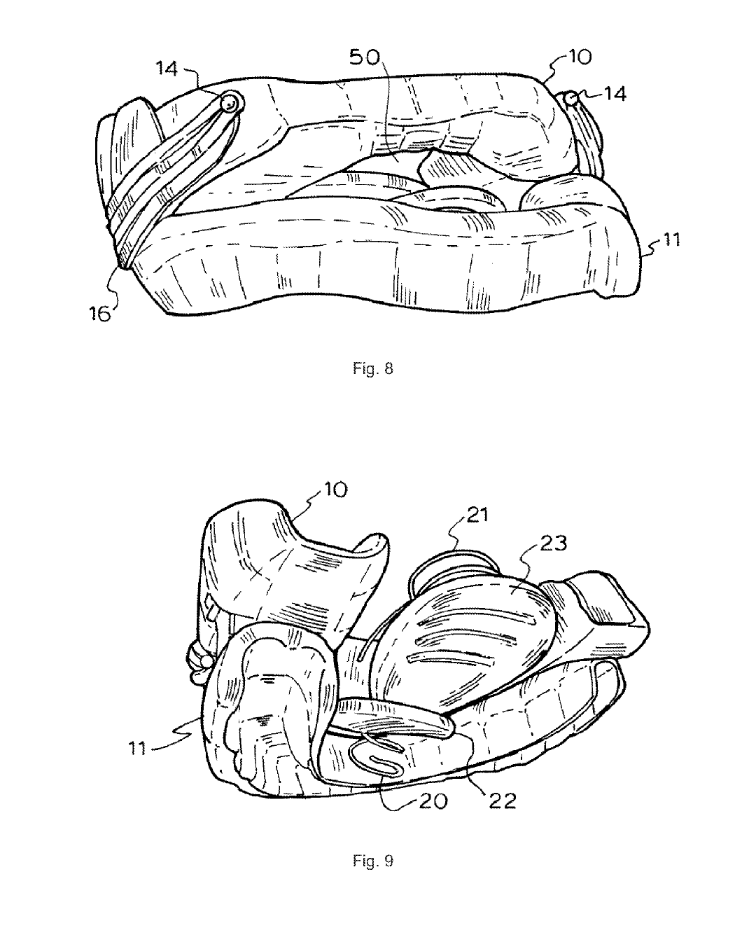

FIG. 2 illustrates the device of FIG. 1, with the tongue 32 shown so as to illustrate the operative positioning of the components. Lower pad 22 fits in front of the base of tongue 32, and it must not extend too far back or could push the tongue backwards to obstruct the airway. A tongue tip space 50 (as will be described further below, see FIG. 8) is provided between the upper and lower carriers, and hence the front teeth, in the operative position.

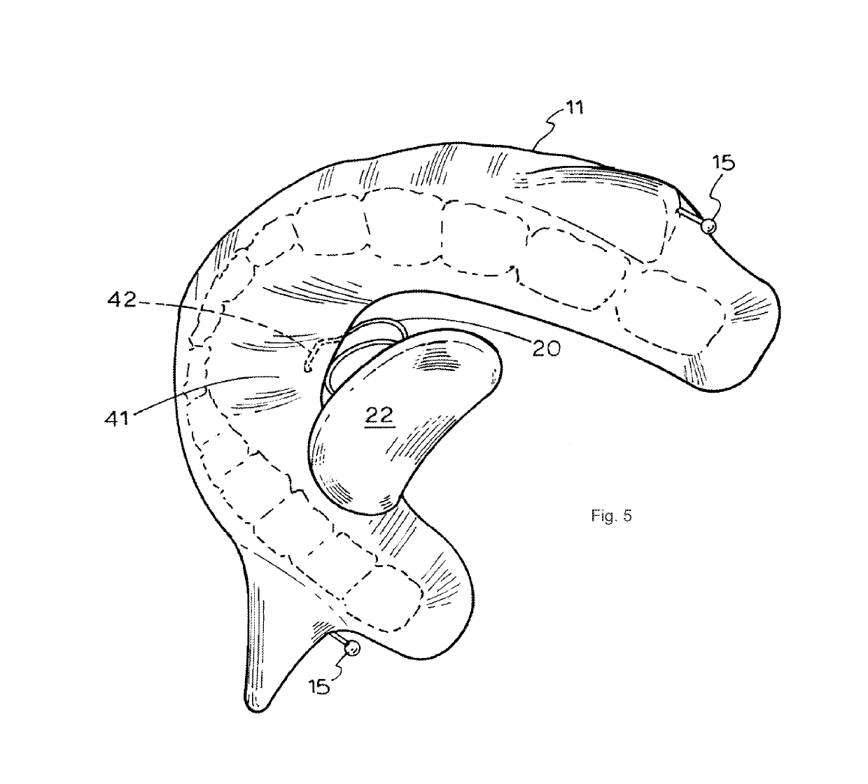

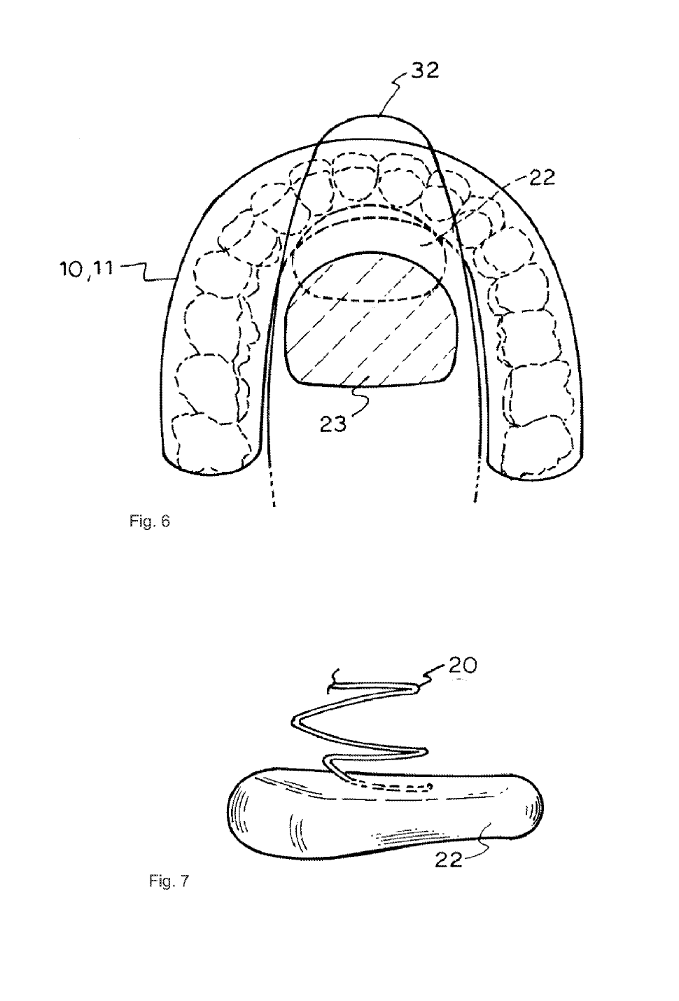

A preferred position for lower pad 22 is just forward of the lingual frenum, so that when tongue 32 is moved forward into the tongue tip space 50, the rear edge of lower pad 22 engages into the lingual frenum thus acting like a pivot or anchor over which the tongue 32 is drawn. This stabilises the tongue and minimises the amount the tongue 32 must protrude past the teeth. Without this pivot/fulcrum the tongue would need to be protruded extra orally past the lips 34, 35, thus degrading patient comfort. The lower pad 22 must be able to move within the sides of the carrier 11 without binding or catching on the surfaces. This can be better seen from FIG. 5, with the interior surfaces labelled generally as 41.

The appropriate size for the pads is determined by the spacing between within the carrier, and hence ultimately by the dimensions of the user's mouth. As discussed, upper and lower pads 22, 23 must be able to move up and down on their respective springs 20, 21 without binding against the sides of the carrier. Further, the pads 22, 23 must be able to be compressed to the extent the spring allows without becoming caught against the sides of the carrier. The surfaces of the pads should be very smooth, without any sharp edges or protrusions. It is preferred that the pads are made from rigid or semi-rigid materials. Soft materials may be used, however, soft materials such as silicone often present issues of fraying and abrasion roughness after prolonged use in mechanically demanding environments such as the present application.

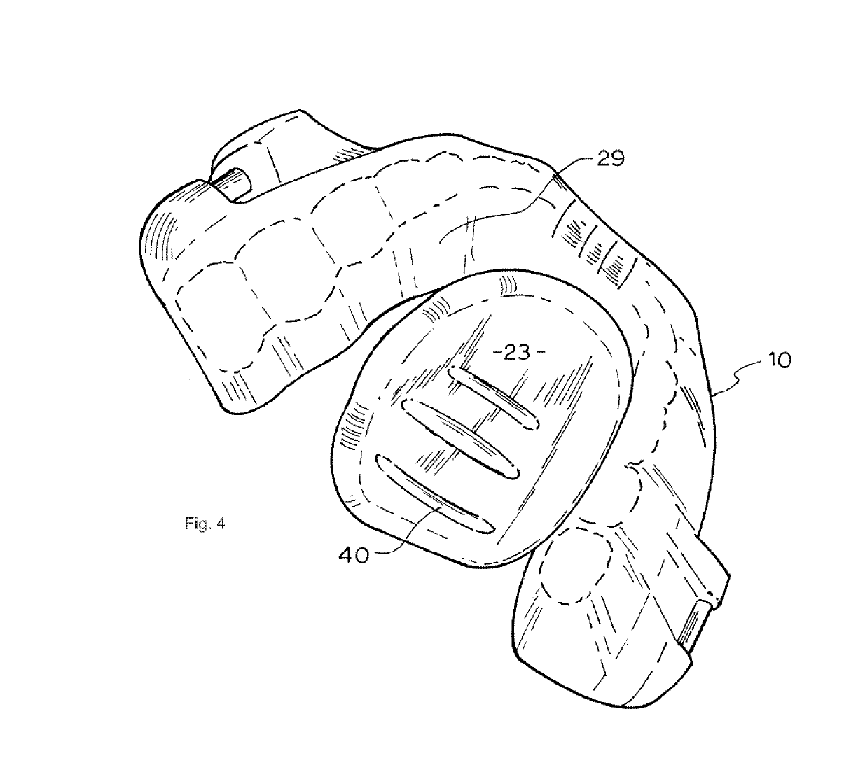

It is preferred that surface texturing, such as waves or similar structures, are provided on the tongue engaging surfaces of the pads. These can be seen, for example, as features 40 in pad 23 in FIG. 4. Pads may be produced without such features. The protruding ridges 40 extend approximately 1-2 mm from the surface of the pad. It will be appreciated that alternative shapes and surface detail may be used. It is important in this implementation that the front edges of both pads have a gentle curve, as they play an important role in the retention of the tongue by pivoting action.

The springs are attached to the pads by any suitable means, preferably by being moulded into the pad as it is formed. For example, in FIG. 7 it can be seen that the spring 20 extends inside the moulded pad 22. Similarly, the spring should be moulded within the carrier, for example as can be seen as feature 43 of FIG. 5. If a cold cure acrylic such as PMMA is used, attachments, repairs and adjustments to the spring can be made using more acrylic, which produces a very strong, integral bond with the carrier and pad. It is preferred that a single point of attachment is used, as this maximises the extent of movement in all directions which can be accommodated by the floating suspension. The preferred attachment point will vary with the attachment mechanism, but needs to be such that the pad is aligned along the central axis of the respective carrier, and towards the front of the mouth, opposing the other pad. The other end of the spring is preferably attached to the respective carrier, to what is the generally vertical surface at the rear of the teeth. It will be appreciated that other attachment points and systems could be used.

Springs 20, 21 are preferably formed from stainless steel, preferably hard stainless steel wire, such as is commonly used in dental applications. It is preferred that the upper spring 21 is formed from 0.7 mm wire, and the lower spring 20 is formed from 0.6 mm wire. For 0.7 mm wire, a coil with a diameter of 15-20 mm is preferred, for a 0.6 mm wire, a coil with a diameter of 10-15 mm is preferred. Generally, only about one and a half turns are present in the spring. It is preferred that the compressive force from each spring, in the neutral position when a tongue is inserted, is sufficient to move the pad into firm engagement with both pads, but not to create an uncomfortable or painful grip. If the springs produce a force which is too great, the tongue will be captured at a particular point, and that area will be crushed. A preferred force applied by each spring is in the range of 1.0 to 2.0 N. It will be understood that this will be adjusted by manipulating the spring at the time of fitting, and by the user. The wire could, if desired, by coated with a biocompatible polymer, with suitable mechanical properties to resist cracking due to fatigue.

The operation of the structure is as follows. The upper and lower carriers 10, 11 are biased together by the respective rubber bands. The upper pad 23 and lower pad 22 are biased inwardly by their respective springs 20, 21. As a consequence, as can be seen in FIG. 2, the tongue 33 is held between the upper pad 23 and lower pad 22. Because of the resilient, floating suspension which is created between the springs, if the tongue moves up or down (i.e. towards the lower or upper teeth), the forces exerted by the springs increase or decrease more or less linearly, so that the tongue continues to be engaged by a more or less constant force between the pads. Further, the nature of the spring structure is that it also allows for some degree of horizontal accommodation of tongue movement.

Equally importantly, the floating suspension also allows for changes in the thickness of the tongue to be readily accommodated, as the springs move the pads into engagement with the tongue as it decreases in thickness or tone. The primary retention is provided by the interaction of the pads with the tongue surface, which particularly includes the effect of the front edge of each pad being pivoted into the tongue as the tongue is withdrawn. This can be seen from FIG. 1—as the tongue is withdrawn in direction 33, the upper pad 21 and lower pad 22 pivot inwardly towards tongue 32 as shown by arrows 25, 26. As the attachment points in this implementation are positioned towards the rear of the pad, the effective retention force is increased as the tongue 32 moves backward, with the pads 21, 22 acting in a similar manner to a soft ratchet to tilt towards and into the surfaces of tongue 32. However, this in no way prevents withdrawal, or has enough force to damage the tongue. The compressive force, the effect of the pad surface and texture, and the tongue-tongue reflex all provide a secondary aspect of retention.

Hence, the tongue can move, and change in shape and tone, while still being retained effectively between the pads. Further, the springs can be manually adjusted as to both position and the force exerted, by increasing or decreasing the length of the spring. As explained above, it is desirable that a voluntary active or involuntary withdrawal of the tongue can be performed in a painless and easy manner. This is readily achieved in a correctly fitted device according to the illustrated implementation of FIG. 1.

An overall device according to this implementation operates by two main mechanisms to relieve OSA. First, the MAS carrier device establishes a forward projecting jaw position, which opens up and stiffens the back of the throat. Second, the pad structure pulls the tongue forward, and prevents it from falling back over the airway. A consequent advantage, relative to existing MAs devices, is that the pad structure actively controls the tongue base position, so as to address another aspect of the OSA process.

A further effect which is exploited by this implementation is the tongue-tongue reflex, which was described above. In FIG. 8, the tongue tip space 50 can best be seen, being the opening between and at the front of the upper 10 and lower 11 carriers. The tongue tip space in front of the pads provides a contact surface which helps to induce this reflex, which in turn will further assist in keeping the tongue forward in the mouth. It is preferred that the tongue tip space be provided with textured features, for example waves, ridges or similar structures, similar in concept to the pad surfaces, to assist in stimulating the tongue tip and thereby instigating the tongue-tongue reflex.

This reflex can also be employed to assist in the operation of the pad structures, particularly the top pad. FIG. 2 illustrates the engagement of the tongue 33 with the upper pad 23. By applying force to the leading edge of the tongue, pad 23 can stimulate the tongue-tongue reflex which will further assist in keeping the tongue in the desired position.

It will be appreciated that the spring could be formed from any suitable, biocompatible, resilient material. Other shapes could be used for the spring, subject to the design avoiding edges and points, so as not to irritate the tongue or other mucous membranes within the mouth. It is important that the spring or other resilient member be adjustable, so as to allow the clinician to make appropriate adjustments, as will be discussed below.

One possible alternative spring structure would use a cantilever spring arrangement. Wires, tensioned and curved to act as cantilever springs, would be attached towards the rear of carriers, and on either side of the pads. It would be possible to use various points on the carrier as attachment points for such springs. Care in the placement of the springs would be required to ensure that any discomfort from tongue or other oral tissue contact with the spring is minimised.

The springs could be replaced with other mechanisms which are able to provide the necessary forces, for example magnetic attraction or repulsion. In such an arrangement, the pad attachment would not be compressive, but rather simply support the pads, with the magnetic force between one or more small magnets placed in each pad providing the necessary engagement with the tongue surfaces. The pad could be attached via a simple swivel type arrangement. In another variation, it would be possible to have the pads both depend from one side, with an appropriate resilient support structure which urges the pads together to provide the floating suspension and buffers or stops to provide the engagement between the pad structure and the other carrier. However, this is likely to be intrusive into the tongue space, and is not preferred. It will be appreciated that combinations of the variations disclosed many be combined as appropriate.

The process of fitting a device to a user according to the preferred implementation is as follows. A dentist takes a top and bottom mouth impression, and then a bite record which registers the correct vertical opening of the jaw, and the degree of protrusion of the jaw. It will be appreciated that an MAS device forms the upper and lower carrier according to this implementation, and that for such a device the lower jaw needs to be correctly protruded relative to the upper jaw. Any such bite record should be taken in accordance with the recommendations of the manufacturer, other than as set out below. The dental laboratory then constructs a standard MAS splint, according to the bite record and impression provided by the dentist. The pads and springs are then fitted to the upper and lower splint, and sized and positioned as described above. The pads may be suitably provided in a small range of fixed sizes, from which the dentist selects the appropriate one having regard to the mouth anatomy of the individual user.

At the time of fitting, the dentist will place the pads into an operative position, by withdrawing them from a compressed position, to make effective contact with each other. The position, and hence force, exerted by each pad and their alignment and positioning is adjusted by the dentist or the user by simple manipulation as required.

For this implementation, it is preferred that the MAS carrier base is prepared in a slightly modified form relative to the standard system. In the case of a Somnomed device, it is preferred that the device is formed so as to have a gap of about 8 to 12 mm between the front teeth when the upper and lower splints are fitted. The appropriate gap will generally be larger in a user with a larger jaw, and smaller in a user with a more gracile jaw. A larger gap is preferred where this is comfortably possible, to provide a larger tongue tip space.

It has been determined by the inventor that providing a larger tongue tip space, relative to prior art MAS devices, provides further advantages for the user. The tongue of the user will locate into the tongue tip space, because the tongue-tongue reflex is stimulated by interaction with the opening. In a further preferred form, the opening is provided with textures features, for example small raised structures such as pimple-type protrusions, waves or curved features, cross hatching, or some other form of texture which will be interesting for the tongue and pleasant for it to repeatedly contact. This assists in keeping the tongue located forward in the mouth, which in turn assists the pads in their objective of keeping the tongue from obstructing the airway during sleep.

The tongue tip space is preferably sufficient to allow the tongue to be protruded over the front teeth and sit just behind the lips. The preferred range of 8 to 12 mm between the front teeth, when allowing for 1 to 2 mm coverage on each side of the teeth, will provide a tongue tip space with a height of 4 to 10 mm. In conventional MAS devices, the tongue is forced to sit behind the closed space of the touching splints at the front of the mouth. This restriction of the tongue space creates an increase in tongue crowding, and can contribute in use to the tongue being forced backwards over the airway. The textured surfaces, particularly on the upper splint surface, provide additional stimulation for the tongue. The textured upper splint surface functions to provide maximal stimulation to the tongue tip, without causing irritation, and so should be smooth and not in any way abrasive.

FIG. 8 illustrates the fitted MAS device which provides the carrier for the pads according to the implementation described above. FIG. 8 is a front view of a MAS splint, modified to increase the tongue tip space 50, and provide ribs to add texture. FIG. 9 is a rear view of such a device. It will be appreciated that in the modified MAS implementation, the pads would not be provided, but as discussed, they could be readily added if required, without the need to make a whole new device.

In a further implementation, the device may be used together with only a resiliently supported lower pad, and no upper pad. It has been observed that for some users, the upper pad may encroach too much into the available tongue space. This then requires a larger vertical opening between the upper and lower supports, in order to accommodate the thereby increased bulk in the tongue space. The larger vertical opening causes an increased tendency (for at least some users) for the tongue to rotate backwards—i.e. towards the rear of the mouth, and this rotation causes degradation of airway potency.

By using the supports plus the lower pad, it is possible to keep jaw opening to a minimum thus minimising this degrading effect.

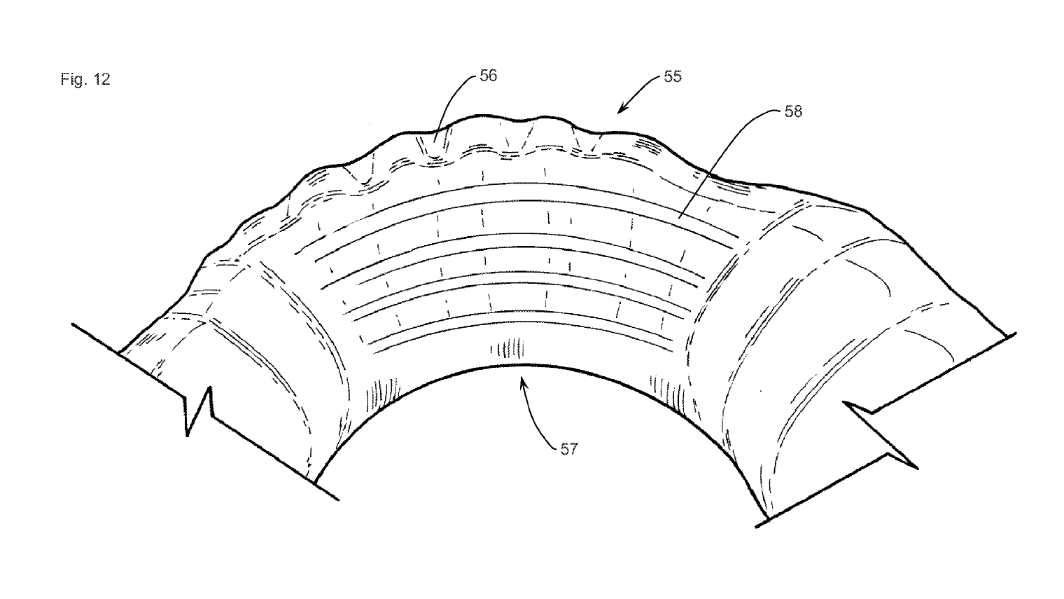

It has been observed in relation to this implementation that operatively the tongue comfortably sits over the lower pad, flowing over it as it relaxes and adhering slightly around the pad, providing a degree of restraint. It has been observed that after continued use, for at least some users, the tongue is trained to just sit in this forward position without needing any other mechanical restraint. In this case, one of the other implementations, for example the integral pad form of FIGS. 11 and 12, may prove to provide adequate outcomes after a period of ‘training’ or acclimation by the tongue.

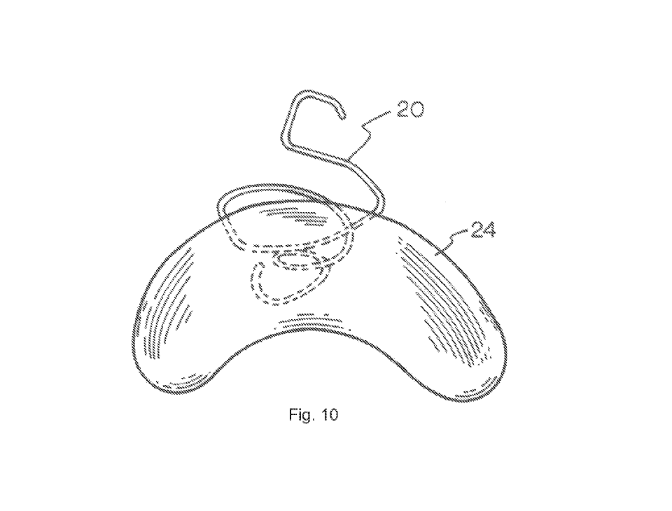

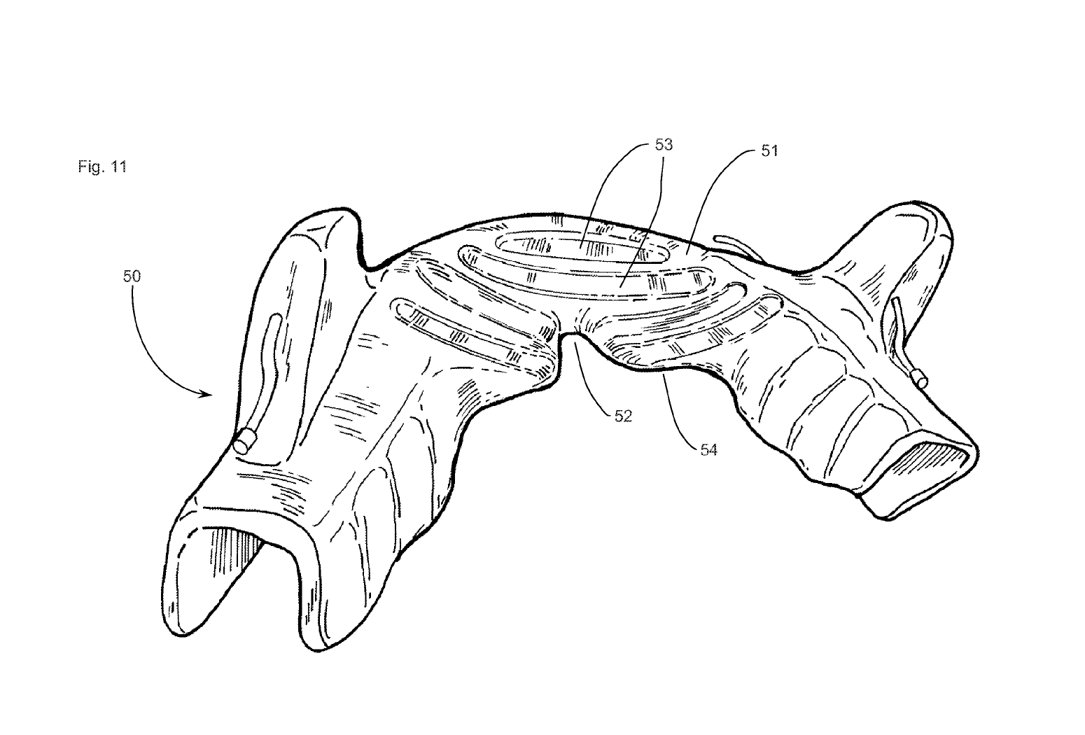

The lower pad may be as shown as pad 22 in FIGS. 1 and 5. It may alternatively have a crescent shape, for example as shown in pad 24 in FIG. 10. It will be appreciated that many alternative shapes and forms may be used. The curve in pad 24 facilitates the pad being, in use, close to and around the lingua frenum under the tongue. In use, in this implementation, the tongue sits with its top surface snugly up against the top support, and the lower surface of the tongue is supported by lower pad 24, with the tongue tip protruding through the through the tongue tip space so it sits just behind the lips.

The pad is supported resiliently, and the spring can preferably be adjusted to adapt to the user and their anatomy and comfort. The resilient suspension allows the pad to follow the tongue's transition in size from awake (firmer, larger) to deep sleep (softer, smaller).

Thus, implementations of the present invention allow for appropriate components of the device to be used as required, in various combinations, in order to provide an acceptable and effective solution for each user. Initially, a generally conventional upper and lower support would be produced, as previously described, and possibly used alone. The supports would have provision for mounting a pad on the lower and upper support if required. If the upper and lower support structure does no provide adequate outcomes, the lower pad could be added. In some cases, the upper pad may be added if required. Whilst in some implementations the upper and lower support would be biased together with elastics, as described above, in other implementations this may not be required. It is a question of the outcomes achieved by individual users, and the alternatives will be able to be trialled in particular patients by those skilled in the art to determine the optimum arrangements. Further, it is envisaged that at the user becomes accustomed to the device, it may be appropriate to vary the configuration to optimise the outcomes.

The inventor has determined that, for at least some users, it may be possible to provide only a MAS type device with an increased tongue tip space, and preferably some texturing inside the space. This device will still achieve effective tongue positioning for some users, and in any case, will improve the effectiveness of an MAS type device. It is believed that the mechanism is that the tongue tip space provides adequate stimulation of the tongue, so that the tongue-tongue reflex is stimulated. In this way, effective tongue position control for at least some users is provided without the need for pads, and it will serve to improve tongue control for MAS users, without a significant increase in costs of the device. The sizing and texturing should be as previously described.

In another aspect, for at least some users, the success may be a consequence of prior training with a device which includes upper and lower pads, as previously described. Accordingly, for at least some users, there may be a process whereby a device with pads is used for a period of time, for example 3 to 6 months, and then a trial is made of the modified device without pads. For at least some users, it will be possible to thereby train the tongue to assume the correct position, and the pads can be removed, or alternatively a device without pads used. In the event that the trial is not successful, the patient can revert to the device with pads. It is noted that the effect may only be temporary, and the user may need to revert to the pads for a period of retraining.

In another variation, the treatment regime could operate as follows. A user presents with OSA, and initially a regular MAS device is made and fitted. In the event that this does not provide adequate relief, the patient has their MAS device modified to add pads, or a new device including pads is provided. The efficacy of this device is then assessed, after a suitable trial period. If it is effective, and well tolerated, the user continues to use this device with pads for a period, for example 3 to 6 months. At the end of that period, the user may at their option trial the device with the pads removed, or another device with no pads, or possibly even revert to the originally fitted device, if it had been modified according to this implementation to include an improved tongue tip space and texturing. If the trial is not successful, the user can revert to the device with pads.

In another implementation, the first device for which the user is fitted includes the enhanced tongue tip space according to this implementation, as well as being constructed so as to permit the ready addition of pads. If this is not proving adequate, the pads are introduced and the patient uses this variant for a period, for example 3 to 6 months. The user may then choose to trial removing the pads. If this is not successful, the pads can be inserted again.

The elastics serve to resiliently retain the upper and lower carriers together, and so define a constant volume within the mouth. This constant volume provides a consistent shape and space for the pads and springs to move within, to allow them to operate in a consistent manner. If the upper and lower carriers are not held in a consistent position, it will be understood that it is difficult for the floating suspension to operate effectively. Whilst the present invention has been predominantly described with reference to a customised, professionally fitted device, the principles of the present invention can also be applied to a more simple device which could be fitted by the user. Such a device could have top and bottom splints, formed from a thermoplastic and joined together with an integral hinge, monobloc style.

The user heats the device in boiling water. Appropriate instructions about the correct protrusion position for the lower jaw can be provided, for example using a link provided to a suitable web based video or any other suitable instruction. The user then bites into the softened device while holding their jaw in the correct position.

The device is preferably formed with a suitable tongue tip space, so as to allow the tongue more room as discussed above. Appropriate texturing could be formed into the device in the tongue tip space. Although such devices are likely to be less effective than a custom device, they could still be useful for relieving snoring and similar conditions, or OSA in some cases. A system of pads for affixing with a suitable mechanism could also be provided. For example, the unmoulded device could be provided in several sizes, with the pre-sized pads appropriate to the interior shapes of each size of device.

It is envisaged that additional features and variations may be added to implementations of the present invention as appropriate. One such variation would be to add more tongue reflex stimulating aspects to the device. For example, being able to vary vertical bite opening in an adjustable, finely controllable way to stimulate the massetter-hypoglossal reflex. This reflex causes tongue protrusion when the massetter jaw muscle is stretched open (in some cases but not in others).

Another addition would be to add more textures or surfaces to stimulate the tongue, particularly the sides of the back third of tongue, to elicit more protrusive reflex response.

Further, the present invention contemplates an implementation in which a mandibular advancement device is enhanced by attaching, for example to a pre-formed attachment feature in the MAD, a tongue support to a lower splint. Thus, if the device is not providing adequate outcomes for the user, the tongue support could be added.

1-35. (canceled)

36. An oral device adapted to operatively enhance the operation of a sleeping user's airway, including a lower carrier adapted to be positioned over at least some of the lower teeth of the user, the lower carrier including a tongue support for operatively supporting the tongue of the user from below, without gripping the tongue, the tongue support being shaped so that in operation it is passively engaged by the tongue of the user.

37. An oral device according to claim 1, wherein the tongue support is located on the device so that operatively the tongue support is positioned forward in the mouth, the tongue support including a set of surface features and textures, such that when the user is asleep, the tongue of the user will tend to locate on the support and thereby locate forward in the mouth.

38. An oral device according to claim 2, wherein the device further includes an upper carrier adapted to be positioned over at least some of the upper teeth of the user, and wherein the upper and lower carriers are operatively adapted to form a mandibular advancement device.

39. An oral device according to claim 2, wherein the tongue support includes a rearward facing recess into which the lingua frenum of the user is operatively received, so that the tongue may rest further forward in the mouth and be further supported along its length from below.

40. An oral device according to claim 1, wherein the upper carrier and lower carrier are dimensioned and shaped so as to operatively provide a tongue tip space into which the user's tongue may locate.

41. An oral device according to claim 5, wherein the tongue tip space includes smoothly textured surfaces on the upper and/or lower carrier, which operatively encourage the tongue to locate in the tongue tip space.

42. An oral device according to claim 5, in which the upper carrier includes a plurality of grooves in the interior surface extending adjacent to the tongue tip space.

43. An oral device according to claim 4, wherein the tongue support is formed so as to generally conform to the shape of the underside of the user's tongue, and include a ramp from the rear, so as to assist in correct location of the tongue.

44. A method for providing support for a tongue in an oral device, so as to operatively enhance the operation of a user's airway, the oral device including a lower carrier adapted to be positioned over at least some of the lower teeth of the user the method comprising providing a tongue support extending from the lower carrier, the tongue support operatively supporting the tongue of the user from below, without gripping the tongue, the tongue support being shaped so that in operation it is passively engaged by the tongue of the user.

45. A method according to claim 9, wherein the device further includes an upper carrier adapted to be positioned over at least some of the upper teeth of the user, and wherein the lower carrier and upper carrier form a mandibular advancement device.

46. A method according to claim 9, wherein the tongue support includes a rearward facing recess into which the lingua frenum of the user is operatively received, so that the tongue may rest further forward in the mouth and be further supported along its length from below.

47. A method for forming an oral device for operatively enhancing the operation of a sleeping user's airway, the device including a lower carrier adapted to be positioned over at least some of the lower teeth of the user, the method comprising providing a tongue support extending from the lower carrier, the tongue support operatively supporting the tongue of the user from below, without gripping the tongue, the tongue support being shaped so that in operation it is passively engaged by the tongue of the user.

48. A method according to claim 11, wherein the tongue support is positioned on the device so as to operatively sit forward in the mouth and includes a set of surface features and textures, such that when the user is asleep, the tongue of the user will tend to locate on the support and thereby locate forward in the mouth.

49. A method according to claim 12, wherein the tongue support includes a rearward facing recess into which the lingua frenum of the user is operatively received, so that the tongue may rest further forward in the mouth and be further supported along its length from below.
